(19) United States
(12) Patent Application Publication
(10) Pub. No.: US 2006/0193891 A1
(43) Pub. Date: Aug. 31, 2006

(54) Medical devices and therapeutic delivery devices composed of bioabsorbable polymers produced at room temperature, method of making the devices, and a system for making the devices

(76) Inventor: Robert Richard, Wrentham, MA (US)

Correspondence Address: KENYON & KENYON LLP, 1500 K STREET N.W., SUITE 700, WASHINGTON, DC, 20005, US

(21) Appl. No.: 11/067,372

(22) Filed: Feb. 25, 2005

(51) Int. Cl. A61F 2/00; B05D 3/00

(52) U.S. Cl. 424/426; 427/2.26

(57) ABSTRACT

A method is provided of making a bioabsorbable appliance that includes selecting a first bioabsorbable polymer having a first glass transition temperature above about room temperature and selecting a second bioabsorbable polymer having a second glass transition temperature below about room temperature. The method also includes combining the first and second bioabsorbable polymers to form a combination and subjecting the combination to a pressure. Additionally, the method includes injecting the combination into a mold in a shape of the bioabsorbable appliance and removing the bioabsorbable appliance from the mold. The method may include adding a bioactive agent to the combination. The steps of combining the first and second bioabsorbable polymers, subjecting the combination to pressure, and injecting the combination into a mold, may be performed at about room temperature. The bioabsorbable appliance may be a stent, a catheter, a guide wire, a balloon, filter, a vena cava filter, a stent graft, a vascular graft, an intraluminal paving system, or an implant. A medical appliance is provided that includes a polymer combination including first and second bioabsorbable polymers formed in a shape of the medical appliance.

### FIELD OF THE INVENTION

The present invention relates to medical devices. More particularly, the present invention relates to a method for making bioabsorbable medical devices at room temperature.

### BACKGROUND INFORMATION

Medical devices may be implanted in human body for various reasons. Medical devices may be coated to provide for the localized delivery of therapeutic agents to target locations within the body, such as to treat localized disease (e.g., heart disease) or occluded body lumens. Localized drug delivery may avoid some of the problems of systemic drug administration, which may be accompanied by unwanted effects on parts of the body which are not to be treated. Additionally, treatment of the afflicted part of the body may require a high concentration of therapeutic agent that may not be achievable by systemic administration. Localized drug delivery may be achieved, for example, by coating balloon catheters, stents and the like with the therapeutic agent to be locally delivered. The coating on medical devices may provide for controlled release, which may include long-term or sustained release, of a bioactive material.

Aside from facilitating localized drug delivery, medical devices may be coated with materials to provide beneficial surface properties. For example, medical devices are often coated with radiopaque materials to allow for fluoroscopic visualization while placed in the body. It is also useful to coat certain devices to achieve enhanced biocompatibility and to improve surface properties such as lubriciousness.

Conventional spray coating stents (such as those described in U.S. Pat. Nos. 4,655,771 and 4,954,126 to Wallsten) may tend to produce coated stents with coatings that are not uniform. U.S. Pat. Nos. 5,824,049 and 6,096,070 to Ragheb et al. mention the use of electrostatic deposition to coat a medical device with a bioactive material. However conventional coating methods may be inefficient and may create uneven and/or non-uniform coatings, thereby affecting the drug release rate for the coated medical appliance.

Medical devices may be designed to incorporate bioabsorbable/biodegradable polymers. The advantage of such materials is that they ultimately are resorbed by the body and therefore may not present long term risks of complications from long-term biological reactions. These devices may be produced using conventional plastics processing techniques such as injection molding or extrusion. While these techniques are effective, biodegradable polymers possess varying degrees of thermal and hydrolytic sensitivity, which may result in dramatic decreases in their molecular weight (and corresponding mechanical properties) during processing. In addition, it is normally not feasible to melt blend many therapeutic materials with polymers due to the inherent thermal instability associated with the therapeutic (small molecule drugs, biologically derived therapeutics such as proteins, DNA, genes cells, etc.).

The publication entitled “Drug Releasing Resorbable Stents With Foam Structure” apparently describes some disadvantages of producing degradable drug eluting devices by high temperature molding, such as loss in molecular weight and degradation of temperature sensitive therapeutics. It is therefore a technical challenge to use thermal molding technology to produce bioabsorbable devices, especially if they are to contain temperature sensitive therapeutics.

The article “Plastics Molded at Room Temperature”, published in the Dec. 1, 2003 edition of the Chemical and Engineering News discusses room temperature processing of polymeric materials. In brief, the technology involves blending a mixture of at least two polymers, with one polymer having a glass transition temperature (Tg) that is at or below room temperature, while the other polymer has a Tg substantially above room temperature. The mixture is subjected to high pressure that is sufficient to cause the mixture to flow at room temperature and allow flow of the mixture to occur.

U.S. Pat. No. 6,503,538 to Chu, et al. relates to an elastomeric functional biodegradable copolyester amides and copolyester urethanes. The Chu reference apparently relates to elastomeric copolyester amides, elastomeric copolyester urethanes, and methods for making the same. The polymers are based on .alpha.-amino acids and possess suitable physical, chemical and biodegradation properties. The polymers are useful as carriers of drugs or other bioactive substances.

U.S. Pat. No. 6,468,519 to Uhrich relates to polyanhydrides with biologically active degradation products. The Uhrich '519 reference apparently relates to polyanhydrides which degrade into biologically active salicylates and alpha-hydroxy acids and methods of using these polyanhydrides to deliver the salicylates and alpha-hydroxy acids to a host.

U.S. Pat. No. 6,486,214 to Uhrich relates to polyanhydride linkers for production of drug polymers and drug polymer compositions produced thereby. The Uhrich '214 reference apparently relates to polyanhydrides which link low molecular weight drugs containing a carboxylic acid group and an amine, thiol, alcohol or phenol group within their structure into polymeric drug delivery systems. Also provided are methods of producing polymeric drug delivery systems via these polyanhydride linkers as well as methods of administering low molecular weight drugs to a host via the polymeric drug delivery systems.

There is, therefore, a need for a simple, cost-effective device for producing a bioabsorbable medical appliance or other device that does not require a high temperature treatment. Each of the references cited herein is incorporated by reference herein for background information.

### SUMMARY

A method is provided of making a bioabsorbable appliance that includes selecting a first bioabsorbable polymer having a first glass transition temperature above about room temperature and selecting a second bioabsorbable polymer having a second glass transition temperature below about room temperature. The method also includes combining the first and second bioabsorbable polymers to form a combination and subjecting the combination to a pressure. Additionally, the method includes injecting the combination into a mold in a shape of the bioabsorbable appliance and removing the bioabsorbable appliance from the mold.

The method may include adding a bioactive agent to the combination.

The step of combining the first and second bioabsorbable polymers to form the combination may include mixing the first and second bioabsorbable polymers.

The method may include inserting the bioabsorbable applicance into a lumen of a body. The method may include contacting the bioabsorbable appliance with a coating.

The steps of combining the first and second bioabsorbable polymers, subjecting the combination to pressure, and injecting the combination into a mold, may be performed at about room temperature.

The bioabsorbable appliance may be a stent, a catheter, a guide wire, a balloon, filter, a vena cava filter, a stent graft, a vascular graft, an intraluminal paving system, or an implant.

The steps of combining the first and second bioabsorbable polymers, subjecting the combination to pressure, and injecting the combination into a mold, may be performed at a process temperature of less than 150 degrees Celsius.

The room temperature may be between about 10 degrees Celsius and about 40 degrees Celsius, and may be about 20 degrees Celsius. The pressure may be at least about 100 psi, may be at least about 200 psi, may be at least about 500 psi, and/or may be at least about 1000 psi.

A medical appliance is provided that includes a polymer combination including first and second bioabsorbable polymers formed in a shape of the medical appliance. The first bioabsorbable polymer has a first glass transition temperature above about room temperature, and the second bioabsorbable polymer has a second glass transition temperature below about room temperature.

The polymer combination may be formed in the shape of the medical appliance by a molding process and/or an extrusion process. The molding process and/or the extrusion process may include applying pressure to the polymer combination.

The medical appliance may be a stent, a catheter, a guide wire, a balloon, filter, a vena cava filter, a stent graft, a vascular graft, an intraluminal paving system, or an implant.

A bioactive agent may be included in the polymer combination.

Room temperature may be defined in this context to be between about 10 degrees Celsius and about 40 degrees Celsius, and may be in particular about 20 degrees Celsius.

The medical appliance may be formed in the shape of the medical appliance at a process temperature of about 150 degrees Celsius.

The pressure may be at least about 100 psi, may be at least about 200 psi, may be at least about 500 psi, and may be at least about 1000 psi.

### BRIEF DESCRIPTION OF THE DRAWINGS

FIG. 1 shows a bioabsorbable stent.

FIG. 2 shows a cross-section of the stent of FIG. 1.

FIG. 3 shows a system for producing a bioaborbable stent.

FIG. 4 shows a flowchart for performing an exemplary method of the present invention.

### DETAILED DESCRIPTION

An exemplary embodiment of the present invention relates to the use of a room temperature molding process to produce bioabsorbable devices such as a bioabsorbable stent. An alternative exemplary embodiment relates to the production of a bioabsorbable article (for instance, a stent) that contains a therapeutic, which may be temperature sensitive.

The process requires a mixture of at least two biodegradable polymers. One polymer has a glass transition temperature (Tg) that is at or below room temperature, and one other polymer has a Tg substantially above room temperature. The mixture is subjected to high pressure that is sufficient to cause the mixture to flow at room temperature and allow flow of the mixture to occur.

Bioabsorbable polymers exist that have Tg's below and above room temperature. Polymers with the right combination of mechanical properties may be selected to meet the Tg requirements detailed above as well as the mechanical properties required for the use intended for the device being designed. By processing the bioabsorbable polymers at room temperature, the kinetic of hydrolysis/degradation would be expected to be much slower than at the high temperatures used for melt processing resulting in improved preservation of the molecular weight and corresponding mechanical properties of the polymers being used. By incorporating one or more therapeutic into the polymer blend it may be possible to make therapeutic delivery devices and at the same time lower the risk of degradation of the therapeutic compared to processes requiring thermal treatment.

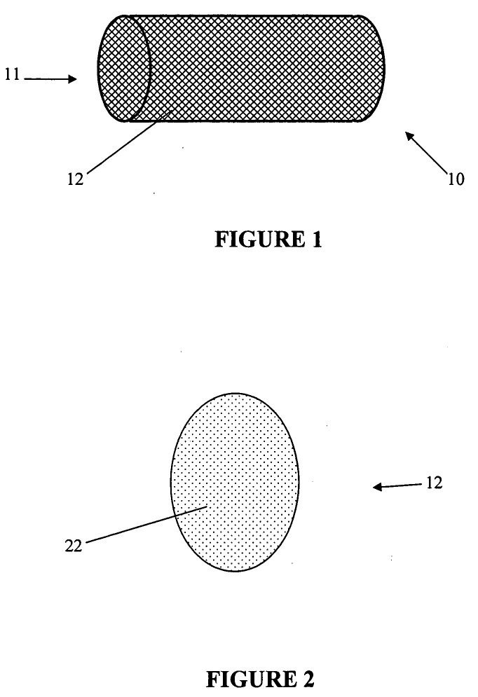

FIG. 1 shows bioabsorbable stent 10, which has interior space 11. Bioabsorbable stent 10 includes struts 12 that are composed of bioabsorbable material. Struts 12, being composed of bioabsorbable materials, may degrade over time after being implanted in a lumen of a human body due to any of heat, hydrolysis, and/or enzymatic reactions. Struts 12 may be a mixture of at least two bioabsorbable polymers. One of the polymers may have a Tg substantially above room temperature (which may be about 20 degrees Celsius), while the other polymer may have a Tg substantially below room temperature. The combination of the two (or more) polymers may flow when subjected to high pressure, and therefore stent 10 may be produced in an injection molding or extrusion process that does not require high temperatures.

FIG. 2 shows a cross-section of strut 12 of the stent of FIG. 1. FIG. 2 shows bioactive agent 22 embedded in the matrix of material of strut 12. Bioactive agent 22 may be any bioactive agent as described herein, and in particular may be a therapeutic that is sensitive to high temperature. Bioactive agent 22 may be released into body tissue or the bloodstream of a human after the stent has been implanted in a human body. Bioactive agent 22 may be released by diffusing out of strut 12 or by the degradation of the matrix material of strut 12, which is bioabsorbable.

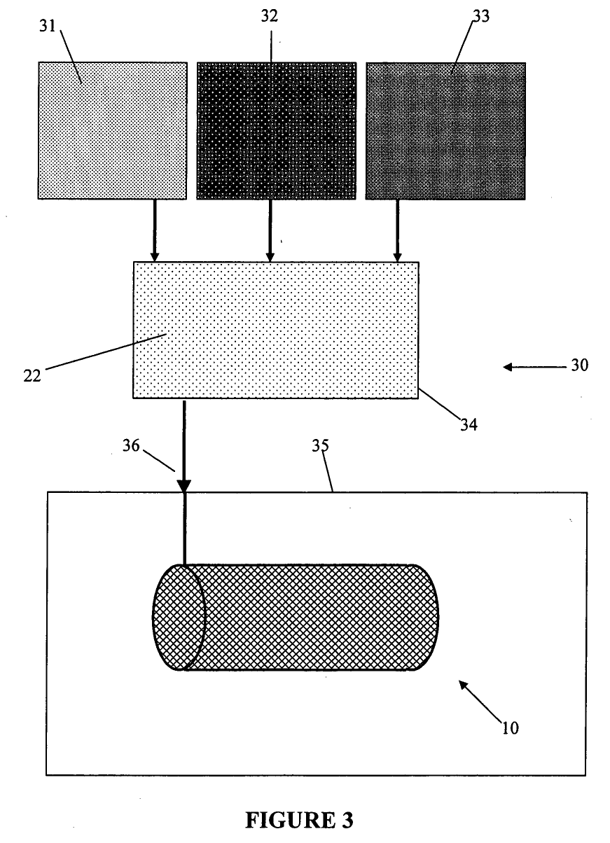

FIG. 3 shows system 30 for producing a bioaborbable stent. System 30 may include several source reservoirs for providing materials to system 30. System 30 of FIG. 3 is shown with three source reservoirs, namely therapeutic source 31, low Tg polymer source 32, and high Tg polymer source 33. High Tg polymer source 33 may include a heating arrangement and/or a pressure arrangement to promote the flow of the high Tg polymer in high Tg polymer source 33. Each of sources 31, 32, 33 feed into mixing container 34. The contents of mixing container therefore include bioactive agent 22, as well as at least two polymers, one polymer having a Tg that is at or below room temperature, and the other polymer has a Tg substantially above room temperature. Mixing container 34 may have an active mixing arrangement, or may allow the materials from sources 31, 32, 33 to mix over time. Mixing container 34 may also be pressurized to promote flowing of the polymer combination. The contents of mixing container 34 may flow through valve 36 into mold 35, which may be an injection mold or an extrusion mold for a medical appliance. As shown in FIG. 3, mold 35 is for producing stent 10, and therefore allows the mixture flowing through valve 36 to fill up a space in mold 35 that replicates the shape of stent 10. Mold 35 may maintain pressure on the mixture flowing through valve 36 until mold 35 if filled by the mixture. Thereafter, valve 36 may be closed and the pressure may be released from mold 35. After waiting an appropriate period for the mixture to solidify in the shape of stent 10, mold 35 may be opened and stent 35 may be removed.

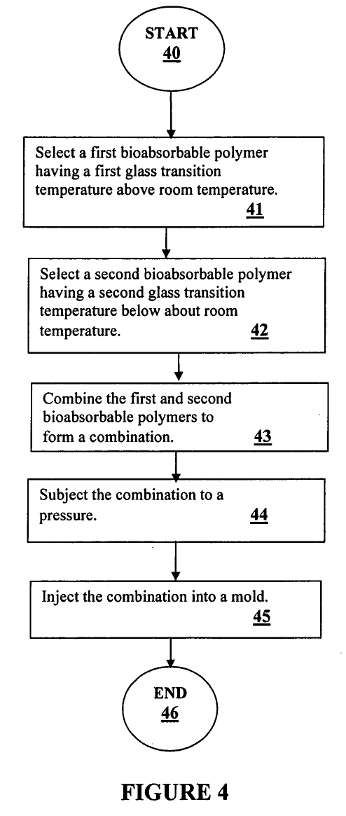

FIG. 4 is a flowchart illustrating an exemplary method of the present invention. The flow in FIG. 4 starts in start circle 40 and proceeds to action 41, which indicates to select a first bioabsorbable polymer having a first glass transition temperature above room temperature. From action 41, the flow proceeds to action 42, which indicates to select a second bioabsorbable polymer having a second glass transition temperature below about room temperature. From action 42, the flow proceeds to action 43, which indicates to combine the first and second bioabsorbable polymers to form a combination. From action 43, the flow proceeds to action 44, which indicates to subject the combination to a pressure. From action 44, the flow proceeds to action 45, which indicates to inject the combination into a mold. From action 45, the flow proceeds to end circle 46.

As used herein, the term “bioactive agent” or “therapeutic agent” includes one or more “therapeutic agents” or “drugs”. The terms “therapeutic agents”, “active substance” and “drugs” are used interchangeably herein and include pharmaceutically active compounds, nucleic acids with and without carrier vectors such as lipids, compacting agents (such as histones), virus (such as adenovirus, andenoassociated virus, retrovirus, lentivirus and α-virus), polymers, hyaluronic acid, proteins, cells and the like, with or without targeting sequences.

The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; inhibitors of heat shock proteins such as geldanamycin; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin-) cells including Lin-CD34−, Lin-CD34+, Lin-cKit+, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable. Non-limiting examples of suitable non-biodegradable polymers include polystrene; polyisobutylene copolymers and styrene-isobutylene-styrene block copolymers such as styrene-isobutylene-styrene tert-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Furthermore, in accordance with various aspects of the present invention, polymeric regions are provided which contain one or more biodisintegrable polymeric phases can be provided using a variety of polymers. Some specific examples include homopolymers and copolymers (e.g., random, statistical, gradient, periodic and block copolymers) that consist of or contain one or more of the following biodisintegrable polymer blocks: (a) biodisintegrable blocks containing one or more biodisintegrable polyesters, including homopolymer and copolymer blocks containing one or more monomers selected from the following: hydroxyacids and lactones, such as glycolic acid, lactic acid, tartronic acid, fumaric acid, hydroxybutyric acid, hydroxyvaleric acid, dioxanone, caprolactone and valerolactone, (b) biodisintegrable blocks containing one or more biodisintegrable polyanhydrides, including homopolymer and copolymer blocks containing one or more diacids such as sebacic acid and 1,6-bis(p-carboxyphoxy) alkanes, for instance, 1,6-bis(p-carboxyphoxy) hexane and 1,6-bis(p-carboxyphoxy) propane; (c) biodisintegrable blocks containing one or more tyrosine-derived polycarbonates/polyarylates, and (d) biodisintegrable blocks containing one or more polyorthoesters, among others.

Some particularly beneficial examples of homopolymers and copolymers include those that consist of or contain one or more biodegradable homopolymer or copolymer blocks that comprise one or more of the following monomers: glycolic acid, lactic acid, caprolactone, trimethylene carbonate, P-dioxanone, hydroxybutyrate, and hydroxyvalerate. Further examples of homopolymer or copolymer blocks include desaminotyrosine polyarylate blocks, desaminotryrosine polycarbonate blocks, polyanhydride blocks such as those formed from therapeutic-based monomers, polyesteramides, and polyetherurethanes. Polyphosphazenes, natural polymers such as carbohydrates, polypeptides/proteins, degradable polyurethanes.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The coating can be applied to the medical device by any known method in the art including dipping, spraying, rolling, brushing, electrostatic plating or spinning, vapor deposition, air spraying including atomized spray coating, and spray coating using an ultrasonic nozzle.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters, guide wires, balloons, filters (e.g., vena cava filters), stents, stent grafts, vascular grafts, intraluminal paving systems, implants and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

While the present invention has been described in connection with the foregoing representative embodiment, it should be readily apparent to those of ordinary skill in the art that the representative embodiment is exemplary in nature and is not to be construed as limiting the scope of protection for the invention as set forth in the appended claims.

What is claimed is:

1. A method of making a bioabsorbable appliance, comprising:
selecting a first bioabsorbable polymer having a first glass transition temperature above about room temperature;
selecting a second bioabsorbable polymer having a second glass transition temperature below about room temperature;
combining the first and second bioabsorbable polymers to form a combination;
subjecting the combination to a pressure;
injecting the combination into a mold in a shape of the bioabsorbable appliance; and
removing the bioabsorbable appliance from the mold.

2. The method of claim 1, further comprising:
adding a bioactive agent to the combination.

3. The method of claim 1, wherein the step of combining the first and second bioabsorbable polymers to form the combination comprises mixing the first and second bioabsorbable polymers.

4. The method of claim 1, further comprising:
inserting the bioabsorbable applicance into a lumen of a body.

5. The method of claim 1, further comprising:
contacting the bioabsorbable appliance with a coating.

6. The method of claim 1, wherein the steps of combining the first and second bioabsorbable polymers, subjecting the combination to pressure, and injecting the combination into a mold, are performed at a process temperature of less than 150 degrees Celsius.

7. The method of claim 1, wherein room temperature is between about 10 degrees Celsius and about 40 degrees Celsius.

8. The method of claim 7, wherein room temperature is about 20 degrees Celsius.

9. The method of claim 1, wherein the pressure is at least about 100 psi.

10. The method of claim 9, wherein the pressure is at least about 200 psi.

11. The method of claim 10, wherein the pressure is at least about 500 psi.

12. The method of claim 11, wherein the pressure is at least about 1000 psi.

13. The method of claim 1, wherein the bioabsorbable appliance is at least one of a stent, a catheter, a guide wire, a balloon, filter, a vena cava filter, a stent graft, a vascular graft, an intraluminal paving system, and an implant.

14. A medical appliance comprising:
a polymer combination including first and second bioabsorbable polymers formed in a shape of the medical appliance;
wherein the first bioabsorbable polymer has a first glass transition temperature above about room temperature; and
wherein the second bioabsorbable polymer has a second glass transition temperature below about room temperature.

15. The medical appliance of claim 14, wherein the polymer combination is formed in the shape of the medical appliance by at least one of a molding process and an extrusion process.

16. The medical appliance of claim 14, wherein the at least one of the molding process and the extrusion process includes applying pressure to the polymer combination.

17. The medical appliance of claim 14, wherein the medical appliance is at least one of a stent, a catheter, a guide wire, a balloon, filter, a vena cava filter, a stent graft, a vascular graft, an intraluminal paving system, and an implant.

18. The medical appliance of claim 14, wherein a bioactive agent is included in the polymer combination.

19. The medical appliance of claim 14, wherein room temperature is between about 10 degrees Celsius and about 40 degrees Celsius.

20. The medical appliance of claim 14, wherein the medical appliance is formed in the shape of the medical appliance at a process temperature of about 150 degrees Celsius.

21. The medical appliance of claim 14, wherein the pressure is at least about 100 psi.

22. The medical appliance of claim 21, wherein the pressure is at least about 200 psi.

23. The medical appliance of claim 22, wherein the pressure is at least about 500 psi.

24. The medical appliance of claim 23, wherein the pressure is at least about 1000 psi.
